(19) United States
(12) Patent Application Publication
(10) Pub. No.: US 2022/0142611 A1
(43) Pub. Date: May 12, 2022

(54) HYBRID MEDICAL IMAGING PROBE, APPARATUS AND PROCESS

(71) Applicant: EMvision Medical Devices Ltd, Brisbane, Queensland (AU)

(72) Inventors: Sasan Ahdi REZAEIEH, Brisbane, Queensland (AU); Ali ZAMANI, Brisbane, Queensland (AU); Amin ABBOSH, Brisbane, Queensland (AU)

(21) Appl. No.: 17/310,890

(22) PCT Filed: Mar. 13, 2020
(86) PCT No.: PCT/AU2020/050242

§ 371 Date: Aug. 27, 2021

(30) Foreign Application Priority Data
Mar. 14, 2019 (AU) 2019900842

(51) Int. Cl. A61B 8/00; A61B 5/0507; A61B 8/08

(52) U.S. Cl. CPC A61B 8/4416; A61B 8/085; A61B 8/4444; A61B 5/0507

(57) ABSTRACT

A hybrid medical imaging probe for application to a body part to image tissues within the body part, the medical imaging probe including: a first imaging probe component to generate non-microwave first signals for transmission into the body part and to sense corresponding signals scattered by the tissues within the body part to enable the generation of one or more corresponding images of the tissues using a non-microwave first imaging technology; and an electromagnetic imaging probe component to generate microwave signals in a microwave frequency band for transmission into the body part and to sense corresponding microwave signals scattered by the tissues within the body part to enable the estimation of corresponding values of permittivity of the tissues; wherein the first imaging probe component and the electromagnetic imaging probe component are co-located within the hybrid medical imaging probe and arranged so that the non-microwave and microwave signals are transmitted from the hybrid medical imaging probe in the same direction.

### TECHNICAL FIELD

The present invention relates to medical imaging, and in particular to a hybrid medical imaging probe apparatus and process for imaging biological tissues of a subject.

### BACKGROUND

Medical imaging technologies such as ultrasound, computed tomography (CT), magnetic resonance imaging (MRI) and nuclear medicine imaging are extremely powerful techniques for imaging internal features of the human body, but suffer from a number of disadvantages that limit their applicability. For example, these technologies require expensive equipment, and are therefore not generally available at rural or remote health centres. Indeed, according to the World Health Organization (WHO), more than half of the world's population does not have access to diagnostic imaging. Furthermore, there is a general need for low-cost and safe imaging systems for the detection and continuous monitoring of a variety of diseases. Due to the need to limit exposure to ionising radiation such as X-rays, most currently available medical imaging systems cannot be used for frequent monitoring purposes. Additionally, the bulky and static structures and high costs of MRI and other large medical imaging systems often preclude them for monitoring diseases that require monitoring on a regular and short-term basis. These factors make such systems impractical to be used by paramedics for real-time imaging and assessment purposes.

Electromagnetic imaging is an attractive technique for medical applications, and has the potential to create a visual representation of the interior of the human body in a cost-effective and safe manner. From an electromagnetic engineering perspective, the human body is an electromagnetically heterogeneous medium characterized by features and tissues with different dielectric properties. Moreover, the dielectric properties permittivity and conductivity differ between injured and healthy tissues. When an injured tissue with a high permittivity value compared to its neighbouring healthy tissue is exposed to an electromagnetic wave at a microwave frequency, a relatively high portion of the wave is reflected back towards the radiation source. Accordingly, an electromagnetic medical imaging apparatus can be utilized to transmit electromagnetic waves into a body part to be imaged, such as the human head or torso. Microwave signals predominantly reflected by damaged tissues (e.g., in particular at bleeding or clot sites) due to changes in electromagnetic properties are received and measured by the apparatus. Then, the data representing the measured signals can be processed to estimate the location and/or dielectric properties of the abnormality, and to generate two or three-dimensional images of the damaged tissues within the body part.

The data processing step plays a critical role in an electromagnetic imaging apparatus. Various imaging techniques have been employed to detect medical targets from measurements of scattered electromagnetic signals. Those techniques try to estimate the dielectric properties of the tissues by solving nonlinear equations (tomography), which do not have a unique solution and those solutions might not depend continuously on the input data, or to find the location of target tissues using time-domain radar-based techniques. Due to the time-consuming nature of tomography-based techniques, they are almost exclusively applicable to single frequency or narrow-band multi-frequency signals, and therefore are not suitable for use in medical emergency situations such as brain injury detection, where a rapid diagnosis is required. Alternatively, in radar-based imaging, a scattering profile of the imaging domain is mapped onto a two- or three-dimensional image. This method is more applicable when using ultra-wide frequency bands for fine resolution because the required data processing is simpler and faster than tomography. However, current radar imaging methods, such as confocal, microwave imaging via space-time (“MIST”) beamforming, and adaptive beamforming imaging methods utilize processing techniques based on delay-and-sum (DAS), which are susceptible to outer layer reflections and internal layer refractions that can result in false detection. In addition, the variation of signal penetration through the tissues at different frequencies limits the effectiveness of those delay calculations, and consequently the accuracy of the resulting images. In view of these difficulties, there is a continuing need for a faster and accurate imaging apparatus and process.

It is desired to overcome or alleviate one or more difficulties of the prior art, or to at least provide a useful alternative.

### SUMMARY

In accordance with some embodiments of the present invention, there is provided a hybrid medical imaging probe for application to a body part to image tissues within the body part, the medical imaging probe including:
a first imaging probe component to generate non-microwave first signals for transmission into the body part and to sense corresponding signals scattered by the tissues within the body part to enable the generation of one or more corresponding images of the tissues using a non-microwave first imaging technology; and
an electromagnetic imaging probe component to generate microwave signals in a microwave frequency band for transmission into the body part and to sense corresponding microwave signals scattered by the tissues within the body part to enable the estimation of corresponding values of permittivity of the tissues;
wherein the first imaging probe component and the electromagnetic imaging probe component are co-located within the hybrid medical imaging probe and arranged so that the non-microwave and microwave signals are transmitted from the hybrid medical imaging probe in the same direction.

In some embodiments, the first imaging probe component is an ultrasonic imaging probe component. In some embodiments, the ultrasonic imaging probe component includes an ultrasonic transducer, and the electromagnetic imaging probe component includes an array of antennas disposed about the ultrasonic transducer.

In some embodiments, the antennas are loaded with series capacitance and/or shunt inductance to create resonances that are independent of the size of the antennas.

In some embodiments, the hybrid medical imaging probe includes electromagnetic bandgap (EBG) structures to reduce the mutual coupling between the antennas, thereby allowing the antennas to be located in close mutual proximity.

In some embodiments, the hybrid medical imaging probe includes artificial magnetic surfaces (AMS) such as metasurfaces formed by arrays of periodic structures and configured so that the array of antennas generate predominantly unidirectional radiation, thereby allowing the antennas to be located in close mutual proximity.

In some embodiments, the hybrid medical imaging probe includes metamaterial absorbers to reduce the leakage of microwave signals.

In accordance with some embodiments of the present invention, there is provided a hybrid medical imaging apparatus for imaging tissues within a body part, the medical imaging apparatus including:
any one of the above hybrid medical imaging probes; and
a data processing component configured to receive initial image data representing an initial image of the tissues of the body part representing non-microwave signals scattered by the tissues within the body part and sensed by the first imaging probe component; and to generate estimates of permittivity of the tissues of the body part based on the sensed microwave signals scattered by the tissues within the body part, wherein the initial image of the tissues of the body part is used as a priori information to generate an electromagnetic model from which the estimates are generated.

In some embodiments, the data processing component is further configured to generate an image representing a spatial distribution of the permittivity of the tissues of the body part.

In accordance with some embodiments of the present invention, there is provided a hybrid medical imaging process for imaging tissues within a body part, the medical imaging process including the steps of:
receiving a first image of the tissues of the body part generated from sensed first and non-microwave signals reflected from the tissues within the body part; and
receiving microwave scattering data representing sensed microwave signals scattered by the tissues within the body part;
processing the first image to generate a corresponding electromagnetic model of the body part; and
processing the microwave scattering data and the electromagnetic model of the body part to generate estimates of permittivity of the tissues of the body part.

In some embodiments, the hybrid medical imaging process includes generating a second image of the tissues of the body part, the second image representing a spatial distribution of the permittivity estimates.

In some embodiments, the first imaging technology is an ultrasonic imaging technology.

In some embodiments, the step of generating the electromagnetic model includes determining a distance between a region of interest within the body part and a corresponding surface of the body part, and an estimate of permittivity of the region of interest is generated by solving a system of equations modelling microwave propagation from the surface to the region of interest and from the region of interest back to the surface of the body part.

In some embodiments, the permittivity value is estimated from scattered microwave signals of a plurality of different microwave frequencies to improve the accuracy of the estimate.

In some embodiments, the tissues include an internal organ, and the process includes assessing a health status of the internal organ from the estimated permittivity value of the internal organ.

In some embodiments, assessing a health status of the internal organ includes estimating a percentage of fat in the internal organ. The internal organ may be a liver.

In some embodiments, the hybrid medical imaging process includes estimating respective permittivities of left and right sides of a patient's torso, and comparing those permittivities to assess a health status of the patient. In some embodiments, assessing a health status of the patient includes diagnosing whether the patient has a disease.

In accordance with some embodiments of the present invention, there is provided at least one computer-readable storage medium having stored thereon executable instructions that, when executed by at least one processor of a data processing apparatus, cause the at least one processor to execute any one of the above processes.

In accordance with some embodiments of the present invention, there is provided a hybrid medical imaging apparatus including:
any one of the above hybrid medical imaging probes; and
any one of the above data processing components.

Also described herein is a medical imaging probe for application to a body part to image tissues within the body part, the medical imaging probe including:
a real-time imaging probe component to generate first signals for transmission into the body part and to sense corresponding signals reflected from the tissues within the body part to enable the generation of one or more corresponding images of the tissues in real-time using a real-time imaging technology; and
an electromagnetic imaging probe component to generate microwave signals in a microwave frequency band for transmission into the body part and to sense corresponding microwave signals reflected from the tissues within the body part to enable the generation of corresponding images of the tissues using a microwave imaging technology.

The real-time imaging probe may be an ultrasonic imaging probe. The ultrasonic imaging probe component may include an ultrasonic transducer, and the electromagnetic imaging probe component may include an array of antennas disposed about the ultrasonic transducer.

Also described herein is a medical imaging apparatus for imaging tissues within a body part, the medical imaging apparatus including:
any one of the above medical imaging probes;
a real-time image generation component to generate an initial image of the tissues of the body part based on the signals reflected from the tissues within the body part and sensed by the real-time imaging probe component; and
an electromagnetic image generation component to generate an electromagnetic image of the tissues of the body part based on the sensed microwave signals reflected from the tissues within the body part, wherein the initial image of the tissues of the body part is used as a priori information to generate the electromagnetic image of the tissues of the body part.

Also described herein is a medical imaging process for imaging tissues within a body part, the medical imaging process including the steps of:
generating a first image of the tissues of the body part based on sensed first signals reflected from the tissues within the body part; and
generating an electromagnetic image of the tissues of the body part based on sensed microwave signals reflected from the tissues within the body part, wherein the accuracy of the generated electromagnetic image is improved by using the first image of the tissues of the body part as a priori information to generate the electromagnetic image, and the first image is generated using a real-time imaging technology.

The real-time imaging technology may be ultrasonic imaging technology.

The step of generating the electromagnetic image may include determining a distance between a region of interest within the body part and a corresponding surface of the body part, and determining a permittivity value for the region of interest by solving a system of equations modelling microwave propagation from the surface to the region of interest and from the region of interest back to the surface of the body part.

Also described herein is a process for diagnosing organ disease in a patient, the process including:
measuring scattering parameters representing electromagnetic signals scattered from organs within a torso of the patient; and
calculating a quantitative measure representing relative permittivity of the organs within right and left sides of the patient's torso; and
diagnosing whether the patient has an organ disease or diffused fat on the basis of a comparison of the quantitative measure with corresponding quantitative measures for the organ in known diseased and healthy states.

### BRIEF DESCRIPTION OF THE DRAWINGS

Some embodiments of the present invention are hereinafter described, by way of example only, with reference to the accompanying drawings, wherein:

FIG. 1 is a prior art ultrasound image that can be used to determine the distance between a patient's skin and their liver;

FIG. 2 is a schematic diagram of a hybrid medical imaging apparatus in accordance with an embodiment of the present invention;

FIG. 3 is a block diagram of a data processing component of the hybrid medical imaging apparatus of FIG. 2;

FIG. 4 is a flow diagram of a hybrid medical imaging process executed by the data processing component of FIG. 3;

FIG. 5 is a schematic diagram of a hybrid electromagnetic-ultrasound probe of the hybrid medical imaging apparatus, in accordance with an embodiment of the present invention; and

FIG. 6 is a schematic diagram illustrating a multilayer dielectric model of the hybrid medical imaging process.

### DETAILED DESCRIPTION

The inventors have identified that the accuracy, speed and reliability of medical electromagnetic imaging (“EM”) can be significantly improved by using a non-microwave first imaging technology to accurately determine the respective locations of one or more targeted tissues or internal organs of a subject (preferably, but not necessarily, in real-time), and then using those locations as a priori information to model microwave propagation to and from the internal organs/tissues and scattering by the internal organs/tissues in order to measure the complex permittivity of those organs/tissues. The permittivity of an internal organ such as the liver is a measure of its health, and can be used to diagnose certain conditions such as fatty liver disease, for example, as described below.

Additionally, the locations of inner organs (or other biological tissue(s) of interest) determined from the first imaging technology can be used to generate corresponding second images of those same tissues or organs using microwave imaging as a second imaging technology (different to the non-microwave first imaging technology), where the second images represent the corresponding spatial distributions of permittivity values.

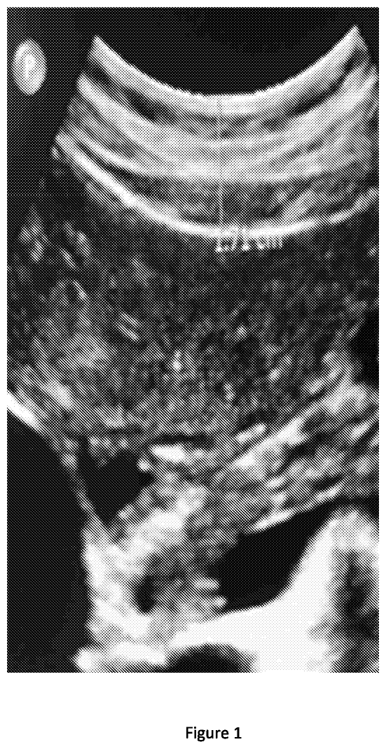

For example, commercially available portable UltraSound (“US”)-machines provide detailed location information of internal tissues and organs using their embedded algorithms, resulting in images such as the one shown in FIG. 1 showing a distance measurement from a patient's skin to an internal organ. Accordingly, embodiments of the present invention include a hybrid medical imaging probe, apparatus and process that combine the benefits of electromagnetic and ultrasonic imaging technologies by using ultrasonic imaging techniques to generate detailed images of internal body tissues of a patient, and then using those ultrasound images as a priori information to estimate dielectric properties and (optionally) to generate corresponding ‘electromagnetic’ images of those same body tissues. However, although some embodiments of the present invention are described herein in the context of combining electromagnetic imaging with ultrasound imaging as the initial imaging technology to generate the prior information, it will be apparent to those skilled in the art that other imaging methods (e.g., sub-millimetre wave imaging) can be used as an alternative to ultrasound imaging in other embodiments.

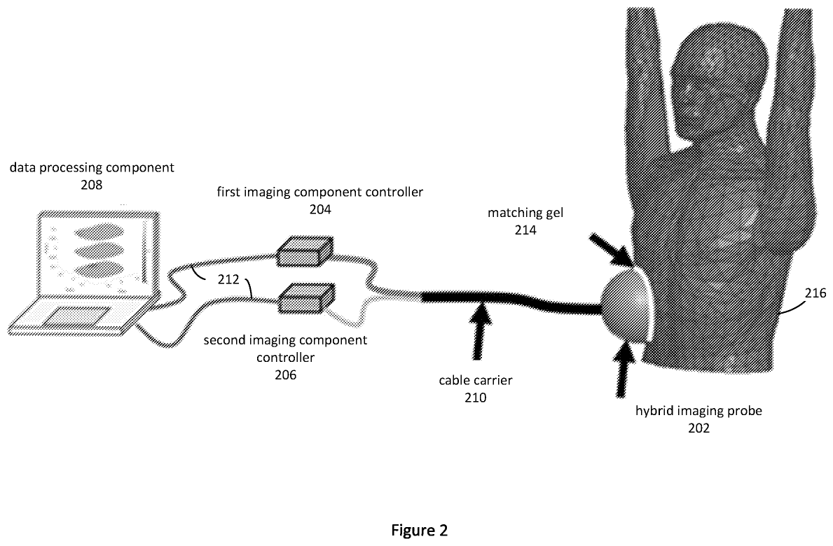

As shown in FIG. 2, a hybrid medical imaging apparatus in accordance with an embodiment of the present invention includes a hybrid imaging probe 202, first and second imaging component controllers 204, 206, and a data processing component 208. In the described embodiments where the first imaging technology is an ultrasound imaging technology, the hybrid imaging probe includes an ultrasound imaging probe component and a microwave imaging probe component, and the first imaging component controller 204 is an ultrasound imaging controller known to those skilled in the art. The second imaging component controller 206 is a microwave imaging component controller, and in the described embodiments is in the form of a vector network analyser (“VNA”) known to those skilled in the art.

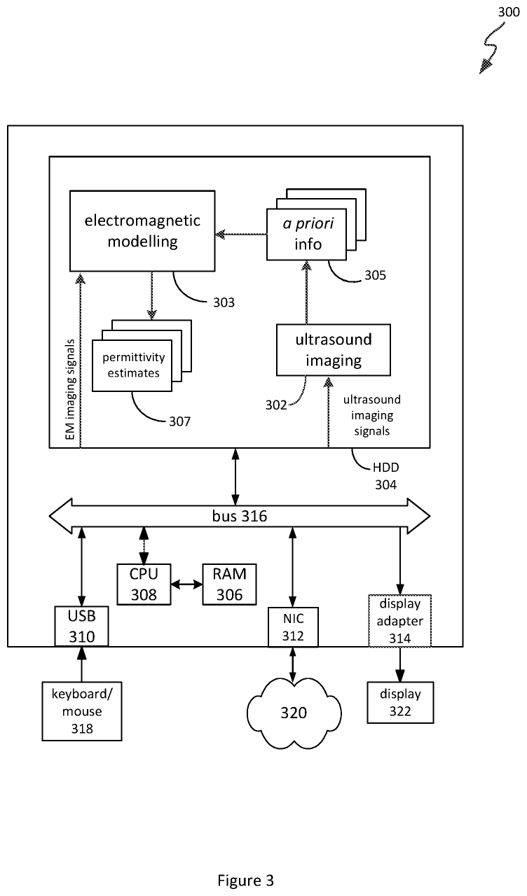

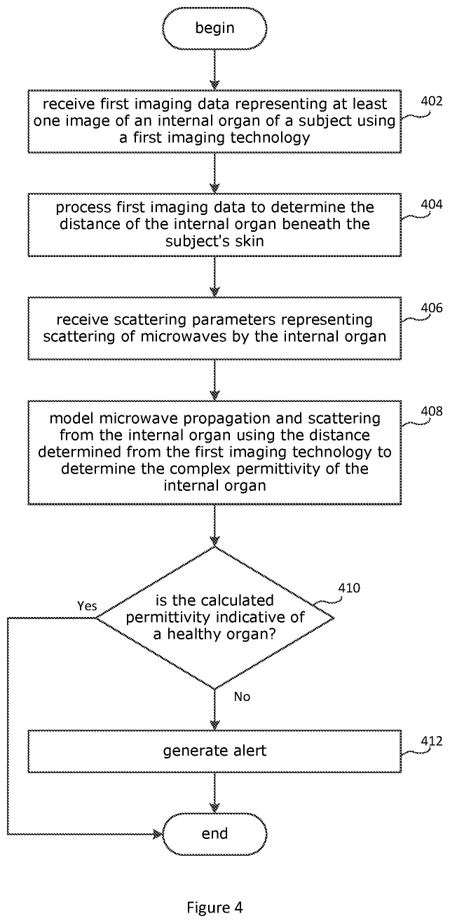

FIG. 3 is a block diagram of the data processing component 208 of the hybrid medical imaging apparatus, in accordance with the described embodiment of the present invention. The data processing component 208 executes a hybrid medical imaging process, as shown in FIG. 4. As indicated in FIG. 2, the data processing component receives imaging data from the first imaging component controller 204 (being an ultrasound imaging component controller in the described embodiments) and electromagnetic (“EM”) scattering data from the second imaging component controller, with both imaging component controllers 204, 206 sending and receiving corresponding signals to and from the hybrid imaging probe 202.

Although the data processing component of the described embodiments is in the form of a computer with hybrid medical imaging processing components 302, 303 installed therein, this need not be the case in other embodiments. As shown in FIG. 3, the data processing component 208 of the described embodiments is based on a 64-bit Intel Architecture computer system, and the hybrid medical imaging process executed by the data processing component 208 is implemented as programming instructions of software components 302, 303 stored on non-volatile (e.g., hard disk or solid-state drive) storage 304 associated with the computer system. However, it will be apparent that at least parts of the hybrid medical imaging process could alternatively be implemented, either in part or in its entirety, in one or more other forms, such as configuration data of a field-programmable gate array (FPGA), and/or as one or more dedicated hardware components, such as application-specific integrated circuits (ASICs), for example.

The data processing component 208 includes random access memory (RAM) 306, at least one processor 308, and external interfaces 310, 312, 313, 314, all interconnected by a bus 316. The external interfaces include universal serial bus (USB) interfaces 310, at least one of which is connected to a keyboard 318 and a pointing device such as a mouse 319, and a display adapter 314, which is connected to a display device such as an LCD panel display 322. The first and second imaging component controllers 204, 206 are communicatively coupled to the data processing component 208 via the USB interfaces 310, allowing these controllers 204, 206 to control their respective probe sub-components.

The software components 302, 303 include a first imaging component 302 that receives imaging signals or data from the first imaging component controller 204, and generates corresponding first images 305 of the subject's tissues. Those first images 305 are then provided as a priori information to an EM processing component 303, which estimates dielectric properties of internal organs/tissues and optionally generates EM images 307 of those organs/tissues from the first images 303 and EM scattering data or signals received from the second (microwave) component controller 206, as described below.

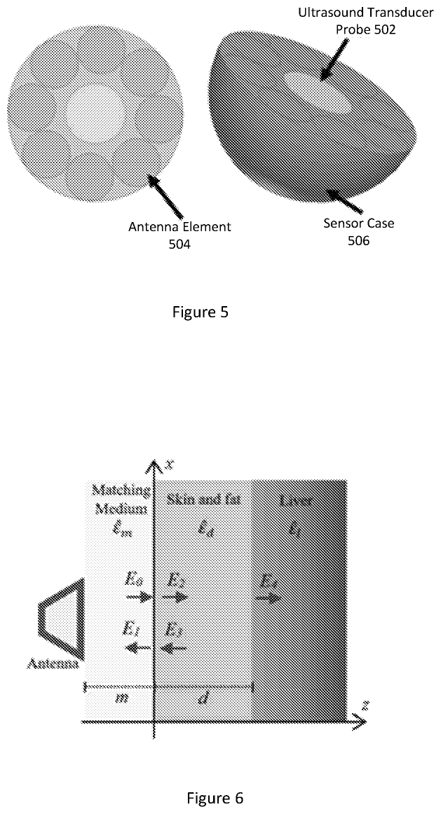

In use, the hybrid electromagnetic-ultrasound (“HEUS”) imaging probe 202 is used to scan a region of interest (e.g., the head or torso) of the body of a subject/patient. As shown in FIG. 5, in the described embodiments the probe 202 includes a wideband antenna or array of antennas 504 that is co-located with an ultrasonic transducer or an array of ultrasonic transducers 502.

Depending on the requirements of the imaging algorithm, the targeted organ to be imaged and the type of images, either an antenna or an array of wideband antennas (as shown in FIG. 5) is used. The size of the antenna(s) and (if an array is used) their mutual coupling can be reduced in several ways, as described below.

For example, in some embodiments, the antenna size is dramatically reduced by applying metamaterial loading in which the antenna is loaded with series capacitance and/or shunt inductance to create resonances that are independent of the size of the antenna, as described in S. Ahdi Rezaeieh, M. A. Antoniades and A. M. Abbosh, “Miniaturization of Planar Yagi Antennas Using Mu-Negative Metamaterial-Loaded Reflector,” IEEE Transactions on Antennas and Propagation, vol. 65, no. 12, pp. 6827-6837, December 2017.

In some embodiments, electromagnetic bandgap (EBG) structures are used to reduce mutual coupling by creating an electromagnetic bandgap that prevents the radiation of surface currents, as described in H. Nakano, K. Kikkawa, N. Kondo, Y. Iitsuka and J. Yamauchi, “Low-Profile Equiangular Spiral Antenna Backed by an EBG Reflector,” IEEE Transactions on Antennas and Propagation, vol. 57, no. 5, pp. 1309-1318, May 2009.

In some embodiments, the antennas include artificial magnetic surfaces (AMS) such as metasurfaces that are formed using arrays of periodic structures to generate unidirectional radiation, as described in A. Rezaeieh, M. A. Antoniades and A. M. Abbosh, “Compact and Unidirectional Resonance-Based Reflector Antenna for Wideband Electromagnetic Imaging,” IEEE Transactions on Antennas and Propagation, vol. 66, no. 11, pp. 5773-5782, November 2018. These surfaces generate zero reflection phase which allows the antennas to be located at close proximity to one another and also to the reflecting surface of the reflector disposed behind each of the antennas.

Finally, in some embodiments, the hybrid probe 202 includes metamaterial absorbers that dissipate the energy of the received signal from certain angles to reduce the leakage of electromagnetic signals from the hybrid probe 202, as required by hospitals.

In the described apparatus, the ultrasound probe component 502 and its corresponding controller 204 are used to provide the prior information regarding the location of the internal tissues or organ (e.g., the liver) of interest relative to the patient's skin. For example, to image the patient's liver, the antenna/antennas transmit microwave signals towards and into the patient's torso, and the reflected signals from each path/tissue are detected and data representing the detected signals sent by the microwave component controller 206 to the data processing component 208. A matching gel 214 can be used between the hybrid probe 202 and the patient's torso to facilitate the penetration of the signals into the patient's body and reduce surface reflections. The antenna and ultrasound signals are transmitted along respective cables by a common cable loom to the hybrid probe 202. The electromagnetic microwave signals are generated and recorded by the portable vector network analyser (VNA) 206. Both the portable VNA 206 and the US-controller 204 are communicatively coupled to the data processing component 208 using suitable data transfer interfaces, cables and protocols, being USB in the described embodiments. The data received from the ultrasound and microwave imaging component controllers 204, 206 are provided as inputs to the hybrid medical imaging process, as described below, and the electromagnetic permittivity and optionally an image of the region of interest is then generated.

In the described embodiments, the scanning domain is modelled as a multilayer dielectric slab which is illuminated by a plane wave normally incident from the or each antenna at z<0, as shown in FIG. 3. The \hat{x}-polarized incident electric field can be expressed as:

Eⁱ(z)=\hat{x}E₀e^{-γm}^{^{z}} (1)

where E₀ is the wave amplitude and γₘ=jω\sqrt{με₀\hat{ε}ₘ} is the propagation constant of the matching medium with complex dielectric permittivity of \hat{ε}ₘ=ε′ₘ−jε″ₘ. The measured distance between the skin and the region of interest, for example the patient's liver d, is used to calculate the total electric field as a function of distance by the sum of traveling waves in each tissue region:

$${E}^{t} ( z ) = \{ \begin{matrix}{E}_{0} {e}^{- {γ}_{{m}^{z}}} + {E}_{1} {e}^{{γ}_{{m}^{z}}} & z < 0 \\ {E}_{2} {e}^{- {γ}_{{d}^{z}}} + {E}_{3} {e}^{{γ}_{{d}^{z}}} & 0 < z < d \\ {E}_{4} {e}^{- {γ}_{l} ( z - d )} & z > d\end{matrix} \qquad ( 2 )$$

Boundary conditions at the interfaces require the continuity of electric and magnetic fields E^{t}(z) and

$$\frac{\partial {E}^{t} ( z )}{\partial z} ,$$

which results in the following equations:

E₀+E₁=E₂+E₃ (3)

E₀−E₁=\hat{n}₂₁(E₂−E₃) (4)

E₂e^{-γ}^{_{d}}^{d}+E₃e^{γ}^{_{d}}^{d}=E₄ (5)

E₂e^{-γ}^{_{d}}^{d}−E₃e^{γ}^{_{d}}^{d}=\hat{n}₃₂E₄ (6)

where,

$${\hat{n}}_{pq} = \sqrt{\frac{{\hat{ɛ}}_{p}}{{ɛ}_{q}}}$$

is the complex refractive index, and \hat{ε}ₚ=ε′ₚ−jε″ₚ is the complex dielectric permittivity of the p-th tissue layer. The solution for the reflected wave is then

$$\begin{matrix}{E}_{1} = \frac{{R}_{32} {e}^{{γ}_{d} d} + {R}_{21} {e}^{- {γ}_{d} d}}{{R}_{21} {R}_{32} {e}^{{γ}_{d} d} + {e}^{- {γ}_{d} d}} {E}_{0} \\ where , & ( 7 ) \\ {R}_{21} = \frac{1 + {\hat{n}}_{21}}{1 - {\hat{n}}_{21}} \\ and & ( 8 ) \\ {R}_{32} = \frac{1 + {\hat{n}}_{32}}{1 - {\hat{n}}_{32}} & ( 9 )\end{matrix}$$

Therefore, the S-parameter measured by the or each antenna is estimated by:

$${\hat{S}}_{11} = \frac{{E}^{t} ( - m )}{{E}^{i} ( - m )} = \frac{{E}_{1} {e}^{- {γ}_{m} m}}{{E}_{0} {e}^{{γ}_{m} m}} = \frac{{E}_{1}}{{E}_{0}} {e}^{- 2 {γ}_{m} m} = \frac{{R}_{32} {e}^{{γ}_{d} d} + {R}_{21} {e}^{- {γ}_{d} d}}{{R}_{21} {R}_{32} {e}^{{γ}_{d} {d}_{+ e} - {γ}_{d} d}} {e}^{- 2 {γ}_{m} m} \qquad ( 10 )$$

In this equation, R₃₂, which is a function of dielectric properties of the liver (in this example), is unknown. Knowing the thickness d and dielectric permittivity of the outer tissue layer \hat{ε}_{d}, as well as the permittivity of the matching medium \hat{ε}ₘ, the unknown parameter R₃₂ is estimated by minimizing the error between the measured and calculated S-parameter, as follows:

$${R}_{32} = arg {min}_{{\hat{ɛ}}_{l}} \vert {S}_{11} - {\hat{S}}_{11} \vert \qquad ( 11 )$$

Because the dielectric permittivity is a complex value, a multi-objective optimization technique (such as the one described in Kaisa Miettinen (1999), Nonlinear Multiobjective Optimization, Springer, ISBN 978-0-7923-8278-2) can be used to find a non-inferior (trade-off) solution for (11) which simultaneously minimises the real and imaginary parts of the error. Therefore, the complex permittivity of the liver \hat{ε}ₗᵢ is estimated by:

$${\hat{ɛ}}_{l} = {( \frac{{R}_{32} - 1}{{R}_{32} + 1} )}^{2} {\hat{ɛ}}_{d} \qquad ( 12 )$$

If the hybrid imaging probe 202 includes an array of antennas, the estimated S-parameters of each element from equation (10) are used to provide an estimation matrix that is used to find the effective permittivity of the liver via an optimization process. In the described embodiments, a distributed iterative optimization algorithm (such as those described in A. Falsone, K. Margellos and M. Prandini, “A Distributed Iterative Algorithm for Multi-Agent MILPs: Finite-Time Feasibility and Performance Characterization”, IEEE Control Systems Letters, vol. 2, no. 4, pp. 563-568, October 2018 and J. Tsitsiklis, D. Bertsekas and M. Athans, “Distributed asynchronous deterministic and stochastic gradient optimization algorithms”, in IEEE Transactions on Automatic Control, vol. 31, no. 9, pp. 803-812, September 1986) is used to minimise the estimation error and converge to the global solution for equation (11). The estimated value is then used in equation (12) to find the effective permittivity \hat{ε}ᵢ of the targeted organ, such as the liver.

In embodiments with wideband or multi-frequency antenna(s), different frequency steps can be used to generate more accurate estimates. In that case, the Debye function is used to model the dielectric permittivity of the targeted tissue according to:

$${\hat{ɛ}}_{l} ( f ) = {ɛ}_{∞} + \frac{{ɛ}_{s} - {ɛ}_{∞}}{1 + j {ωτ}_{0}} \qquad ( 13 )$$

where, εₛ is the permittivity at zero frequency, ε_{∞} is the permittivity at infinite frequency, and τ₀ is the relaxation time. By substituting equation (13) in the refraction index formula and solving the optimization problem of equation (11) for the three constants εₛ, ε_{∞}, and τ₀, the dielectric properties of the organ, such as the liver, can be estimated as a function of frequency. In that regard, the signals should be sampled evenly and the number of frequency samples should be greater than six (twice the number of unknowns in the Debye function of equation (13)).

Knowing the values of the permittivity and conductivity of the healthy organ, such as the liver, across the used frequency band, the difference between the estimated permittivity of the scanned patient's organ, such as the liver, and the healthy organ can be interpreted to assess the healthy or unhealthy status of the organ, such as finding the percentage of fat in the liver for the case of fatty liver disease, for example.

In some embodiments, a horizontal cross-section of a patient's chest (torso) is scanned and virtually divided into two portions representing the “right side” and “left side” of the patient's torso so that the right side portion is mainly occupied by the patient's liver, whereas the left side portion of contains the patient's spleen, pancreas and kidney organs. In the microwave frequency band of 0.5-1 GHz, the dielectric properties of the organs on the left side have an average permittivity of 60, whereas the average permittivity of a healthy liver is about 48. Thus, there is about a 25% difference between the dielectric properties of the left and right-side organs in a healthy patient. Accordingly, the inventors have determined that, using the signal processing techniques described herein, the amplitude and phase of the back scattered microwave signals that are reflected or transmitted through these organs on the left and right side portions of the patient's torso can be used to determine the permittivity of the investigated organ. Then, these calculated values are used to define a threshold/range for healthy subjects. That is, if a person is healthy, then the reflected/transmitted signals from left and right sides exhibit a difference of around 25%. However, the average permittivity of fatty liver tissue is around 37, which increases the ratio of the signals for the left and right sides to about 62%, and there is more than 100% contrast between the permittivity of livers of healthy and unhealthy persons. Thus, these values can be used to diagnose and monitor fatty liver and similar diseases in the chest area.

Many modifications will be apparent to those skilled in the art without departing from the scope of the present invention.

1. A hybrid medical imaging probe for application to a body part to image tissues within the body part, the medical imaging probe including:
a first imaging probe component to generate non-microwave first signals for transmission into the body part and to sense corresponding signals scattered by the tissues within the body part to enable the generation of one or more corresponding images of the tissues using a non-microwave first imaging technology; and
an electromagnetic imaging probe component to generate microwave signals in a microwave frequency band for transmission into the body part and to sense corresponding microwave signals scattered by the tissues within the body part to enable the estimation of corresponding values of permittivity of the tissues;
wherein the first imaging probe component and the electromagnetic imaging probe component are co-located within the hybrid medical imaging probe and arranged so that the non-microwave and microwave signals are transmitted from the hybrid medical imaging probe in the same direction.

2. The hybrid medical imaging probe of claim 1, wherein the first imaging probe component is an ultrasonic imaging probe component.

3. The hybrid medical imaging probe of claim 2, wherein the ultrasonic imaging probe component includes an ultrasonic transducer, and the electromagnetic imaging probe component includes an array of antennas disposed about the ultrasonic transducer.

4. The hybrid medical imaging probe of claim 3, wherein the antennas are loaded with series capacitance and/or shunt inductance to create resonances that are independent of the size of the antennas.

5. The hybrid medical imaging probe of claim 3, including electromagnetic bandgap (EBG) structures to reduce the mutual coupling between the antennas, thereby allowing the antennas to be located in close mutual proximity.

6. The hybrid medical imaging probe of claim 3, including artificial magnetic surfaces (AMS) such as metasurfaces formed by arrays of periodic structures and configured so that the array of antennas generate predominantly unidirectional radiation, thereby allowing the antennas to be located in close mutual proximity.

7. The hybrid medical imaging probe of claim 3, including metamaterial absorbers to reduce the leakage of microwave signals.

8. A hybrid medical imaging apparatus for imaging tissues within a body part, the medical imaging apparatus including:
the hybrid medical imaging probe of claim 1; and
a data processing component configured to receive initial image data representing an initial image of the tissues of the body part representing non-microwave signals scattered by the tissues within the body part and sensed by the first imaging probe component; and to generate estimates of permittivity of the tissues of the body part based on the sensed microwave signals scattered by the tissues within the body part, wherein the initial image of the tissues of the body part is used as a priori information to generate an electromagnetic model from which the estimates are generated.

9. The hybrid medical imaging apparatus of claim 8, wherein the data processing component is further configured to generate an image representing a spatial distribution of the permittivity of the tissues of the body part.

10. A hybrid medical imaging process for imaging tissues within a body part, the medical imaging process including the steps of:
receiving first image data representing a first image of the tissues of the body part generated from sensed non-microwave signals scattered by the tissues within the body part;
receiving microwave scattering data representing sensed microwave signals scattered by the tissues within the body part;
processing the first image to generate a corresponding electromagnetic model of the body part; and
processing the microwave scattering data and the electromagnetic model of the body part to generate estimates of permittivity of the tissues of the body part.

11. The hybrid medical imaging process of claim 10, including generating a second image of the tissues of the body part, the second image representing a spatial distribution of the permittivity estimates.

12. The hybrid medical imaging process of claim 10, wherein the first imaging technology is an ultrasonic imaging technology.

13. The hybrid medical imaging process of claim 10, wherein the step of generating the electromagnetic model includes determining a distance between a region of interest within the body part and a corresponding surface of the body part, and an estimate of permittivity of the region of interest is generated by solving a system of equations modelling microwave propagation from the surface to the region of interest and from the region of interest back to the surface of the body part.

14. The hybrid medical imaging process of claim 13, wherein the permittivity value is estimated from scattered microwave signals of a plurality of different microwave frequencies to improve the accuracy of the estimate.

15. The hybrid medical imaging process of claim 10, wherein the tissues include an internal organ, and the process includes assessing a health status of the internal organ from the estimated permittivity value of the internal organ.

16. The hybrid medical imaging process of claim 15, wherein assessing a health status of the internal organ includes estimating a percentage of fat in the internal organ.

17. The hybrid medical imaging process of claim 10, including estimating respective permittivities of left and right sides of a patient's torso, and comparing those permittivities to assess a health status of the patient.

18. The hybrid medical imaging process of claim 17, wherein assessing a health status of the patient includes diagnosing whether the patient has a disease.

19. At least one computer-readable storage medium having stored thereon executable instructions that, when executed by at least one processor of a data processing apparatus, cause the at least one processor to execute the process of claim 10.

20. A hybrid medical imaging apparatus including:
the hybrid medical imaging probe of claim 1; and
a data processing component configured to execute the process of claim 10.
